(19) United States
(12) Patent Application Publication
(10) Pub. No.: US 2009/0069764 A1
(43) Pub. Date: Mar. 12, 2009

(54) CONNECTOR FOR STOMA BAG, OF THE TWO-PIECE TYPE, FIXED BY GLUING

(75) Inventors: Delphine Burlot, Anglet (FR); Paul Lassalle, Biarritz (FR)

Correspondence Address: YOUNG & THOMPSON, 209 Madison Street, Suite 500, ALEXANDRIA, VA, 22314, US

(73) Assignee: B. BRAUN MEDICAL SAS, Boulogne-Billancourt (FR)

(21) Appl. No.: 12/278,447

(22) PCT Filed: Feb. 16, 2007
(86) PCT No.: PCT/FR2007/000286

§ 371 Date: Aug. 6, 2008

(30) Foreign Application Priority Data
Feb. 17, 2006 (FR) 0601424

(51) Int. Cl. A61F 5/449

(52) U.S. Cl. 604/345

(57) ABSTRACT

A connector for a stoma bag, of the two-piece type, fixed by gluing includes a support equipped with a bracket for fixing to the skin of a patient and with a gluing channel, and a bag provided with a gluing channel that carries an adhesive. The adhesive in the gluing channel of the bag is a layer of a structural adhesive having a thickness of at least 0.6 mm, which forms an elastomeric adhesive composition. The adhesive of the structural adhesive layer contains a three-sequence copolymer, such as a styrene-isoprene-styrene copolymer. In the channel of the bag, its surface carrying the adhesive has undergone a treatment to increase adherence, such as a treatment to increase the surface roughness. The connector can include a member for firstly positioning the channel of the bag relative to the channel of the support. The connector applies also to colostomy, ileostomy and urostomy bags.

This invention relates to a two-part-type ostomy appliance. More specifically, the invention relates to an adhesive connector for such an appliance.

An adhesive connector for a two-part-type ostomy appliance comprises, on the one hand, a support that is equipped with a clamp for attaching to a patient's skin and a bonding strip, and, on the other hand, a bag that is equipped with a bonding strip bearing an adhesive. These bonded connectors allow the use of the same support with several successive bags, without irritating the patient's skin by frequent removal of the support. Since the adhesive that attaches the bag to the support is the interface element, it constitutes the most fragile element and, for this reason, it is always located on the bag, which is the element for single use and short duration, and not on the support, which is a relatively permanent element, used with several bags one after the other.

In these two-part-type appliances with bonded connectors, the adhesive that is located between the strips on the support and on the bag, for not remaining bonded to the strip on the support during the removal of the bag, always has a greater adhesion to the strip on the bag than to the strip on the support.

The use of such a two-part-type ostomy appliance therefore comprises the implementation of two bonded interfaces: a first interface is found between the support and the patient's skin, and the other between the strips on the support and on the bag.

These bonded interfaces comprise adhesives that are both of the “structural” type. A “structural” adhesive is such that the bonded parts support a load that is at least equal to their weight. These adhesives are distinguished from holding adhesives that are not continuously subjected to detachment forces, and plugging adhesives whose function is essentially to form an obturation. In the case of ostomy appliances, when a bag contains evacuated materials, its weight is much higher than that of parts bonded by an adhesive that should therefore be of the structural type.

It is known that structural adhesives are stronger, the smaller their thickness, taking into account the limits set by the application. Actually, excessive thickness of the film or of the adhesive layer reduces the holding force; however, it is necessary that the film or the layer not be too thin, because it can then create a discontinuous bonding. In the case of ostomy appliances, any discontinuity of bonding leads to leaks of the evacuated materials, in particular liquids and gases (bad odors).

The adhesive layers of the ostomy appliances should have a particularly high shear resistance because, in a patient's standing position, all of the weight of the bag is exerted in the plane of the bonding surfaces.

In a general way, it is considered that, in the bonded joints, the structural adhesive should have a thickness of between 0.1 and 0.43 mm (4 to 13 thousandths of an inch). For this reason, for the bonding of strips on the support and on the bag, a structural adhesive with a small thickness, on the order of 0.1 to 0.3 mm in general, is used.

However, the structural adhesive layer that is located between the support and the patient's skin has a greater thickness, for example 1 to 2 mm. Actually, the use of a simple adhesive in contact with the skin poses problems of cutaneous tolerance, such that it is not possible to use a “simple” adhesive. The material that is used, on the one hand, should be deformable, taking into account movements of the surface of the skin (a rigid layer greatly reduces the comfort of the patient) due to movements of the patient, but should also have non-irritating properties upon contact with the skin. For this reason, essentially “gum” layers loaded with hydrocolloids are used. The “structural” resistance of such a layer is obtained by the use of a material with elastomer properties.

The structural adhesive layer that is located between the support and the patient's skin is therefore specially adapted to the conditions that are set by this application: the deformation of the skin and, in a more general way, the body part surrounding an ostomy when the patient carries out various movements, and the absence of skin irritation. These conditions are met during the use of a “gum” layer with elastomer properties loaded with hydrocolloids, having a thickness on the order of one to two millimeters.

In the two-part-type ostomy appliances with bonded connector in question, the gum layer, most often bonded to a support sheet (this layer and the sheet, sometimes with a protective sheet of the gum, are sometimes called “clamp” or “cutaneous protector”) is in general attached to the strip on the support by a simple circular weld of small width between the support sheet and the strip. In this way, the strip on the support is relatively independent of the clamp, and the strip on the bag can thus be easily bonded to the strip on the support.

However, it has already been noticed, as described in, for example, the document WO 96/38 106, that sometimes folds are formed between the strips on the support and on the bag, in particular when the patient who is wearing the appliance sits down. The concept is that such folds, if they lead to the formation of channels, i.e., more or less fine separations along one or more fold lines, will almost necessarily cause leaks not only of gas and odors in the case of colostomy bags, but primarily fluids in the case of ileostomy and urostomy bags.

The formation of these folds, and therefore separations of the strip from the bag, is due to the fact that the strip on the support and, consequently, that of the bag after bonding are not detached adequately from the clamp of the support that should have a great capacity for deformation and great flexibility to follow the movements of the patient as well as possible.

To solve this problem, the documents WO-01/85 074 and WO-2004/039 293 describe various solutions that are designed to increase the decoupling between the strip on the support and the clamp of the latter.

The invention relates to an entirely different solution of the above-mentioned problem posed by the formation of folds that may be causing leaks.

More specifically, according to the invention, the adhesive layer that ensures the bonding between the strip on the support and the strip on the bag consists of a mass with elastomer properties that is thick enough that it absorbs the deformations at one or more fold lines and prevents in practice the leaks between the two strips.

Thus, the invention relates to an adhesive connector for a two-part-type ostomy appliance, comprising a support that is equipped with a clamp for attachment to a patient's skin and a bonding strip, and a bag that is equipped with a bonding strip that bears an adhesive; according to the invention, the adhesive of the bonding strip of the bag is a layer of a structural adhesive that has a thickness of at least 0.5 mm and preferably at least 0.6 mm.

Preferably, the structural adhesive layer of the bag forms an adhesive elastomer mass.

Preferably, the adhesive of the structural adhesive layer contains a polymer that is selected from among the urethane polymers and the sequenced triple-sequence copolymers. The sequenced triple-sequence copolymer is, for example, selected from among the copolymers styrene-isoprene-styrene, styrene-butadiene-styrene, styrene-ethylene-butadiene-styrene, and styrene-isoprene-butadiene-styrene. The sequenced copolymer can also be mixed with a linear polymer.

Preferably, the surface of the strip of the bag bearing the adhesive has undergone a treatment for increasing adhesion. For example, the treatment for increasing adhesion is a chemical treatment, such as a surface substitution reaction or a physical treatment, such as a treatment for increasing micro-roughness, for example by a treatment by effluvia.

In one variant, the adhesive layer is bonded to an intermediate support sheet itself bonded to the strip of the bag.

Preferably, the connector comprises a prepositioning element of the strip on the bag relative to the strip on the support. In one example, the prepositioning element comprises cooperation elements located outside of the strips on the support and on the bag.

In another example, the prepositioning element is located inside the strips on the bag and on the support, and the adhesive layer of the strip on the bag bears a protective sheet that is formed by at least two parts that are folded on themselves.

Owing to the above-mentioned properties, the strip complex of the support/structural adhesive/strip on the bag can be folded to an angle of 135° before separations form.

Thus, according to the invention, deformation properties are introduced into the adhesive layer that is located between the two strips. These deformability properties are obtained by use of a particular material with elastomer properties, having, of course, adhesive properties with regard to the strips on the support and on the bag.

The material that is used for this purpose and that has elastomer properties is advantageously a sequenced copolymer. The sequenced copolymer can be a styrene-isoprene-styrene copolymer, a styrene-butadiene-styrene copolymer, a styrene-ethylene-butadiene-styrene copolymer, or a styrene-isoprene-butadiene-styrene copolymer. This sequenced copolymer can be mixed with other polymers, in particular of the linear type, for example a polymer of one of the sequenced copolymer groups in question. The main thing is that the material that is obtained for the layer has elastomer properties.

The adhesive of the structural adhesive layer can also be a polyurethane-based gum.

Of course, as in any connector for a two-part-type ostomy appliance, the adhesive layer should exhibit a greater adhesion to the strip on the bag than to the strip on the support.

This result can be obtained in various known ways. First of all, the chemical natures of the two strips can be selected so that the material of the strip on the bag is more compatible with the material of the adhesive layer than the material of the strip on the support.

The relative adhesions of the adhesive layer with two strips can be modulated either by increasing the adhesion to the strip on the bag or by reducing the adhesion to the strip on the support. Of course, it is preferable to obtain a high adhesion and therefore to increase the adhesion to the strip on the bag.

The increase in adhesion can be obtained by a chemical treatment or by a physical treatment.

As a chemical treatment, it is possible to consider a chemical treatment of the surface by a substitution reaction of the surface groups of the strip material or by a simple chemical attack.

As a physical treatment, it is possible to consider, in a known way, the increase in roughness of the surface of the strip on the bag, for example by creating a micro-roughness by various physical processes, either purely mechanical or electric, for example by discharges by effluvia.

One example of adhesion reduction treatment with the strip on the support can be, in contrast, a smoothing of the surface of the strip on the support, for example by use of a material whose surface has undergone an at least partial fusion.

When achieving this adhesion gradation poses problems, it is possible to use an intermediate sheet. More specifically, the adhesive layer with elastomer properties can be bonded to a thin sheet with which it is very compatible, and this thin sheet is itself bonded by a very thin structural adhesive to the strip on the bag, Such adhesives can be those that are commonly used with a small thickness between the strips of known two-part appliances.

As in any bonded connector for a two-part-type ostomy appliance, it is desirable that the two connector parts be positioned in advance relative to one another, before the bonding. For this purpose, it is advantageous that the connector comprise a prepositioning element for example of the type described in the document WO-2005/025466.

It is necessary to note that the structural adhesive of the bag bonding strip is usually cut into a sheet. It is not unusual that such a sheet, for example formed by calendaring or extrusion, has local thickness variations, often of at least 10%. The given values for the thickness of the structural adhesive layer of the bag can therefore vary in a corresponding manner.

### EXAMPLE

In one example, the strip on the bag is formed by polyethylene with a 450 μm thickness, having a tensile strength of 49 N. The strip on the support is formed by ethylene copolymer and vinyl acetate with a 350 μm thickness, having a tensile strength of 38 N. Its elastic limit is 20 N.

The material of the structural adhesive layer with elastomer properties is a styrene-isoprene-styrene copolymer that has a Shore hardness A of less than 8, a Young's module of less than 0.06 MPa, and a thickness of 0.9 mm.

The surface of the strip on the bag has undergone a treatment by effluvia, such that it has a peeling separation force that is clearly higher than 38 N, whereas the peeling separation force from the strip on the support is on the order of 23 N.

In one variant, the material of the structural adhesive layer with elastomer properties is a urethane polymer (PUR) that has a Shore hardness A of less than 15, a Young's module of less than 0.05 MPa, and a thickness of 0.9 mm.

1. Connector for an adhesive ostomy appliance, of the two-part type, comprising a support that is equipped with a clamp for attaching to a patient's skin and a bonding strip, and a bag that is equipped with a bonding strip bearing an adhesive, wherein the adhesive of the bonding strip of the bag is a structural adhesive layer that has a thickness of at least 0.5 mm.

2. Connector according to claim 1, wherein the structural adhesive layer of the bag forms an elastomer adhesive mass.

3. Connector according to claim 2, wherein the adhesive of the structural adhesive layer contains a polymer that is selected from among the urethane polymers and the sequenced triple-sequence copolymers.

4. Connector according to claim 3, wherein the sequenced copolymer is selected from among the following copolymers: styrene-isoprene-styrene, styrene-butadiene-styrene, styrene-ethylene-butadiene-styrene and styrene-isoprene-butadiene-styrene.

5. Connector according to claim 1, wherein the strip on the bag has a surface that bears the adhesive that has undergone an adhesion-increasing treatment.

6. Connector according to claim 5, wherein the physical treatment for increasing adhesion is a treatment for increasing roughness.

7. Connector according to claim 1, wherein the adhesive layer is bonded to an intermediate support sheet that is itself bonded to the strip on the bag.

8. Connector according to claim 1, wherein it comprises a prepositioning element of the strip on the bag relative to the strip on the support.

9. Connector according to claim 8, wherein the prepositioning element comprises cooperation elements located outside of the strips on the support and on the bag.

10. Connector according to claim 8, wherein the prepositioning element is located inside the strips on the bag and on the support, and the adhesive layer of the strip on the bag bears a protective sheet formed by at least two parts that are folded on themselves.

11. Connector according to claim 2, wherein the strip on the bag has a surface that bears the adhesive that has undergone an adhesion-increasing treatment.

12. Connector according to claim 3, wherein the strip on the bag has a surface that bears the adhesive that has undergone an adhesion-increasing treatment.

13. Connector according to claim 4, wherein the strip on the bag has a surface that bears the adhesive that has undergone an adhesion-increasing treatment.

14. Connector according to claim 2, wherein the adhesive layer is bonded to an intermediate support sheet that is itself bonded to the strip on the bag.

15. Connector according to claim 3, wherein the adhesive layer is bonded to an intermediate support sheet that is itself bonded to the strip on the bag.

16. Connector according to claim 4, wherein the adhesive layer is bonded to an intermediate support sheet that is itself bonded to the strip on the bag.

17. Connector according to claim 2, wherein it comprises a propositioning element of the strip on the bag relative to the strip on the support.

18. Connector according to claim 3, wherein it comprises a prepositioning element of the strip on the bag relative to the strip on the support.

19. Connector according to claim 4, wherein it comprises a prepositioning element of the strip on the bag relative to the strip on the support.
